(19) United States
(12) Patent Application Publication
(10) Pub. No.: US 2022/0110996 A1
(43) Pub. Date: Apr. 14, 2022

(54) MOISTURIZING COMPOSITION

(71) Applicant: Wanda Lewis, Russellville, AR (US)

(72) Inventor: Wanda Lewis, Russellville, AR (US)

(21) Appl. No.: 17/068,072

(22) Filed: Oct. 12, 2020

(51) Int. Cl. A61K 36/889; A61K 9/00; A61K 36/88; A61K 31/355; A61K 9/06; A61K 45/06; A61M 31/00

(52) U.S. Cl. CPC A61K 36/889; A61K 9/0034; A61K 36/88; A61M 2210/1475; A61K 9/06; A61K 45/06; A61M 31/00; A61K 31/355

(57) ABSTRACT

A moisturizing composition, the composition comprising a sufficient amount of extra-virgin coconut oil, a sufficient amount of aloe barbadensis, and a sufficient amount of vitamin E oil. The composition is provided as a cream, which may be applied to a body cavity, such as within and/or around the vagina.

### TECHNICAL FIELD

The embodiments generally relate to moisturizing compositions and, more particularly, to moisturizing compositions which can be delivered to a body cavity.

### BACKGROUND

Moisturizing compositions, such as hand lotions and body lotions, have been used to moisturize the skin and promote healing if the skin is dry or cracked; however, many of these compositions are not suitable for application inside body cavities or to open wounds. For example, women are vulnerable to tearing and/or disease within the genital tract as the lining of the vagina is a permeable mucous membrane. In many cases, pregnancy and delivery of an infant, intercourse, lack of lubrication during intercourse, and changes in the cervix during the menstrual cycle may affect the permeable vaginal wall and cause complications.

The vaginal cavity provides a unique site for the delivery of therapeutic agents; however, several factors must be taken into consideration when developing therapeutic compositions and delivery systems for body cavities, and specifically for those applied to the vagina. For example, the vaginal anatomy, mucosal surface, the presence and composition of vaginal fluids and secretions, cervical fluids, cyclical changes, and the microflora must be considered.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide a moisturizing composition comprising a sufficient amount of extra-virgin coconut oil, a sufficient amount of aloe barbadensis, and a sufficient amount of vitamin E oil. The composition is provided as a cream which may be applied to a body cavity, such as within and/or around the vagina.

The moisturizing composition is optimized for application to bodily cavities, such as the vagina, is formulated to alleviate discomfort, vaginal dryness, itching, pain, and promote healing of the vaginal tissues and surrounding areas, and may be applied one or more times throughout the day, prior to or following intercourse, as well as prior to or following vaginal delivery. The moisturizing composition provides continual vaginal and/or labia hydration and lubrication. The moisturizing composition may also be utilized as a prophylactic for any of the above ailments.

In one aspect, the sufficient amount of aloe barbadensis is provided as a mixture comprising suitable amounts of at least one of the following: agar, citric acid, absorbic acid, and potassium sorbate.

In one aspect, the sufficient amount of vitamin E oil is provided as a mixture comprising suitable amounts of at least one of the following: sunflower extract, sweet almond seed extract, apricot oil, jojoba oil, rosehip seed oil, lavender oil, argan kernel oil.

In one aspect, the moisturizing composition is applied one or more times daily.

In one aspect, the moisturizing composition is applied prior to, during, or after intercourse.

In one aspect, the moisturizing composition is suitable to ease symptoms related to itchiness.

In one aspect, the moisturizing composition is suitable to ease symptoms related to dryness.

### DETAILED DESCRIPTION

The specific details of the single embodiment or variety of embodiments described herein are to the described composition and method of use thereof. Any specific details of the embodiments are used for demonstration purposes only, and no unnecessary limitations or inferences are to be understood therefrom.

In general, the embodiments provided herein relate to a moisturizing composition optimized for application to bodily cavities, such as the vagina. The moisturizing composition is formulated to alleviate discomfort, vaginal dryness, itching, pain, and promote healing of the vaginal tissues and surrounding areas and may be applied one or more times throughout the day, prior to or following intercourse, prior to or following vaginal delivery, etc. The moisturizing composition provides continual vaginal and/or labia hydration and lubrication.

In some embodiments, the composition is formulated as a cream, which may be applied to the vaginal tissues and/or surrounding areas. However, the composition may also be formulated in various configurations for topical use, including solutions, suspensions, creams, ointments, salves, gels, aerosols, medicated bandages and gauze, emulsions, douches, washes, and surfactants in general.

In some embodiments, the moisturizing composition is formulated for multiple applications each day to alleviate vaginal dryness, itching, and pain, and to promote healing of the vaginal tissues and surrounding areas.

The composition comprises a sufficient amount of each of the following: organic extra-virgin coconut oil, organic aloe barbadensis, agar, citric acid, absorbic acid, potassium sorbate, a vitamin E oil, sunflower extract, sweet almond seed extract, apricot oil, jojoba oil, rosehip seed oil, lavender oil, and argan kernel oil.

In some embodiments, the organic aloe barbadensis, agar, citric acid, absorbic acid, and potassium sorbate are preformulated as a mixture.

In some embodiments, the organic vitamin E oil, sunflower extract, sweet almond seed extract, apricot oil, jojoba oil, rosehip seed oil, lavender oil, and argan kernel oil are preformulated as a mixture.

In some embodiments, the extra-virgin coconut oil is provided as a moisturizer which can be applied within the body cavity or surrounding areas to provide relief from dryness, itchiness, and discomfort.

In some embodiments, the extra-virgin coconut oil is provided as 100% pure cold pressed and unrefined coconut oil.

Vitamin E is known to reduce itchiness by moisturizing the skin, vaginal tissues, and surrounding areas to provide relief from itchiness. Further the antioxidant properties of vitamin E may prevent or reduce the side effects of various conditions.

In one exemplary formulation of the composition including sufficient amounts of the components, the composition is comprised of about 27 parts organic extra-virgin coconut oil, about 3 parts mixture of organic aloe barbadensis, agar, citric acid, absorbic acid, potassium sorbate, and about 1 part mixture of organic vitamin E oil, sunflower extract, sweet almond seed extract, apricot oil, jojoba oil, rosehip seed oil, lavender oil, argan kernel oil. In other words, the composition is about 87% organic extra-virgin coconut oil, about 10% mixture of organic aloe barbadensis, agar, citric acid, absorbic acid, and potassium sorbate, and about 3% mixture of organic vitamin E oil, sunflower extract, sweet almond seed extract, apricot oil, jojoba oil, rosehip seed oil, lavender oil, argan kernel oil.

In another exemplary formulation of the moisturizing composition, the composition comprised between about 70-90% organic extra-virgin coconut oil, between about 5-20% mixture of organic aloe barbadensis, agar, citric acid, absorbic acid, and potassium sorbate, and between about 0.1%-10% mixture of organic vitamin E oil, sunflower extract, sweet almond seed extract, apricot oil, jojoba oil, rosehip seed oil, lavender oil, argan kernel oil.

In some embodiments, the moisturizing composition is provided during production into an airless pump bottle configured to emit about ¼ teaspoon of the composition with each pump of the airless pump bottle. The airless pump bottle may be any such container known in the arts which is capable of storing and emitting the moisturizing cream composition.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A moisturizing composition, comprising:
a sufficient amount of extra-virgin coconut oil;
a sufficient amount of aloe barbadensis; and
a sufficient amount of vitamin E oil,
wherein the composition is provided as a cream, and wherein the composition is suitable for application to a body cavity.

2. The moisturizing composition of claim 1, wherein the sufficient amount of aloe barbadensis is provided as a mixture comprising suitable amounts of at least one of the following: agar, citric acid, absorbic acid, and potassium sorbate.

3. The moisturizing composition of claim 1, wherein the sufficient amount of vitamin E oil is provided as a mixture comprising suitable amounts of at least one of the following: sunflower extract, sweet almond seed extract, apricot oil, jojoba oil, rosehip seed oil, lavender oil, argan kernel oil.

4. The moisturizing composition of claim 1, wherein the moisturizing composition is suitable for application one or more times daily.

5. The moisturizing composition of claim 1, wherein the moisturizing composition is suitable for application prior to, during, and after intercourse.

6. The moisturizing composition of claim 1, wherein the moisturizing composition is suitable to ease symptoms related to itchiness.

7. The moisturizing composition of claim 1, wherein the moisturizing composition is suitable to ease symptoms related to dryness.

8. A moisturizing composition, comprising:
about between 70-90% extra-virgin coconut oil;
about between 5-20% aloe barbadensis; and
about between 0.1%-10% vitamin E oil,
wherein the composition is provided as a cream, and wherein the composition is suitable for application to a body cavity.

9. The composition of claim 8, wherein the extra-virgin coconut oil is pure, coldpressed and unrefined extra-virgin coconut oil.

10. The composition of claim 9, wherein the aloe barbadensis is organic aloe barbadensis.

11. The composition of claim 10, wherein the vitamin E oil is organic vitamin E oil.

12. The composition of claim 11, wherein the cream is provided in an airless pump bottle.

13. The composition of claim 12, wherein the airless pump bottle is configured to emit ¼ teaspoon of the moisturizing composition per pump.

14. The moisturizing composition of claim 13, wherein the sufficient amount of aloe barbadensis is provided as a mixture comprising suitable amounts of at least one of the following: agar, citric acid, absorbic acid, and potassium sorbate.

15. The moisturizing composition of claim 14, wherein the sufficient amount of vitamin E oil is provided as a mixture comprising suitable amounts of at least one of the following: sunflower extract, sweet almond seed extract, apricot oil, jojoba oil, rosehip seed oil, lavender oil, argan kernel oil.

16. The moisturizing composition of claim 15, wherein the moisturizing composition is suitable for application one or more times daily.

17. The moisturizing composition of claim 16, wherein the moisturizing composition is suitable for application before, during, and after intercourse.

18. The moisturizing composition of claim 17, wherein the moisturizing composition is suitable to ease symptoms related to itchiness.

19. The moisturizing composition of claim 18, wherein the moisturizing composition is suitable to ease symptoms related to dryness.

20. A moisturizing composition, comprising:
about between 70-90% extra-virgin coconut oil;
about between 5-20% a aloe barbadensis a mixture comprising at least one of the following: agar, citric acid, absorbic acid, and potassium sorbate; and
about between 0.1%-10% a vitamin E oil a mixture comprising at least one of the following: sunflower extract, sweet almond seed extract, apricot oil, jojoba oil, rosehip seed oil, lavender oil, argan kernel oil,
wherein the composition is provided as a cream, wherein the cream is suitable to be emitted in ¼ teaspoon portions from an airless pump bottle.
